(19) United States
(12) Patent Application Publication
(10) Pub. No.: US 2013/0324437 A1
(43) Pub. Date: Dec. 5, 2013

(54) METHOD FOR IDENTIFYING ANTIMICROBIAL COMPOUNDS AND DETERMINING ANTIBIOTIC SENSITIVITY

(71) Applicants: Joseph Pogliano, San Diego, CA (US); Kit Pogliano, San Diego, CA (US)

(72) Inventors: Joseph Pogliano, San Diego, CA (US); Kit Pogliano, San Diego, CA (US)

(73) Assignee: THE REGENTS OF THE UNIVERSITY OF CALIFORNIA, Oakland, CA (US)

(21) Appl. No.: 13/816,471

(22) PCT Filed: Aug. 12, 2011
(86) PCT No.: PCT/US11/47582

§ 371 Date: Aug. 16, 2013

Related U.S. Application Data
(60) Provisional application No. 61/373,144, filed on Aug. 12, 2010.

(51) Int. Cl. C12Q 1/18; C12Q 1/68

(52) U.S. Cl. CPC C12Q 1/18; C12Q 1/68; C12Q 1/689
USPC 506/10; 435/6.1; 435/32; 435/6.15

(57) ABSTRACT

Provided herein are improved methods of characterizing antibiotic agents according to the effect the antibiotic agent has on a bacterial cell. This allows for rapid screening of potential antibiotic agents, e.g., for improved antibiotics, and classification of antibiotic agents. The present techniques also allow for profiling of bacterial strains for susceptibility of antibiotic agents and, e.g., determining which are most susceptible to particular modes of action.

### CROSS-REFERENCES TO RELATED APPLICATIONS

This application claims priority to U.S. Ser. No. 61/373,144, filed Aug. 12, 2010, the disclosure of which is incorporated by reference in its entirety.

### BACKGROUND OF THE INVENTION

The market for many individual antibiotics is greater than $1 billion. Many methods are available for identifying antibacterial compounds. The oldest and still most commonly used approach is to screen for compounds that inhibit growth of bacteria (e.g., in liquid culture or semisolid media such as agar). Alternative approaches include phenotypic screening, in vitro screening, and structure-based drug design. Phenotypic screening methods involve searching for compounds that inactivate a particular gene or pathway essential for cell viability, e.g., using a GFP or β-galactosidase reporter system. In vitro screens employ high throughput enzyme assays to find drugs that inhibit a specific enzyme. Candidate drugs from these screens are then typically tested in phenotypic screens or growth inhibition assays. In rational drug design, a limited number of compounds that are predicted to interact with the target are tested for the ability to inhibit a particular enzyme using in vivo and in vitro methods.

These approaches ultimately rely on population-based bioassays to report the ability of a candidate drug to interfere with bacterial growth. They typically require relatively large populations of bacteria, and large amounts of both candidate drugs, and known control drugs. These population-based methods are slow, as they require multiple generations of cell growth, and provide no insight into the mechanism of action (MOA) of the candidate antibacterial. Bacterial cells are relatively difficult to kill. Agents that result in harmed or defective cellular physiology often are not detected by standard growth curves. Bacteria with defects in important cellular processes (e.g., DNA replication or cell division) appear to be identical to wild type in population-based cell growth assays. For example, using assays for cell growth (e.g., colony forming units, optical density readings, or minimal inhibitory concentration (MIC)), bacteria grow in sublethal concentrations of antibiotics appear similar to untreated controls. These assays are thus not sufficiently sensitive for detecting subtle effects of antibacterial drugs.

Cytological profiling has been used to compare the effects of drugs on eukaryotic cells (Perlman & Slack (2004) Science 306:1194; Mitchison (2005) Chembiochem 6:33; Feng et al. (2009) Nat Rev Drug Discov 8:567). Bacteria, however, were long believed to be bags of enzymes with little internal organization, based on more than five decades of bacterial research. Bacteria do not contain a nucleus, golgi apparatus, spindle pole bodies, mitochondrion, microtubules, or other subcellular compartments that were the basis for cytological profiling in eukaryotes. This general approach was therefore thought to be inapplicable to bacteria cells. As noted above, bacteria have traditionally been studied using general survival and growth assays. The field did not recognize that subcellular features could be detected and used to characterize bacteria and bacterial responses to various stimuli and antibiotics.

### BRIEF SUMMARY OF THE INVENTION

Provided herein are specific cytological assays, and combinations thereof that can be used for high throughput screening of antibiotic agents. These methods allow for characterization and classification of antibiotics. Cytological profiling can also be used for identifying bacteria in a sample, e.g., based on cytological profiles of known bacterial strains, and for determining the antibiotic susceptibility of particular strains of bacteria. The methods are broadly applicable, and can be used to characterize compounds directed against any microorganism, including gram positive bacteria, gram negative bacteria, mycobacteria, and mycoplasma.

Provided herein are methods for determining the mechanism of action (MOA) of an antibiotic agent (known or potential) comprising contacting the antibiotic agent with a microorganism; detecting the effect of the antibiotic agent on at least one cytological characteristic of the microorganism; comparing the effect of the antibiotic agent on the at least one cytological characteristic to that of a control (or more than one control); and determining the MOA of the antibiotic agent. In some embodiments, the microorganism is bacteria (e.g., gram positive or gram negative). In some embodiments, the control is the microorganism (the same or different species or strain of the microorganism) not contacted with the antibiotic agent. In some embodiments, the control is the microorganism (the same or different species or strain of the microorganism) contacted with an antibiotic agent with a known MOA. In some embodiments, the control represents an average value for the cytological characteristic of a plurality of antibiotic agents with a known MOA, or an average value for the cytological characteristic obtained from more than one species or strain of microorganism. In some embodiments, the control is represented by the microorganism separately contacted with a panel of antibiotic agents with different, known MOAs.

In some embodiments, the at least one cytological characteristic is selected from the group consisting of cell size (e.g., length, width, or volume); chromosome shape (e.g., ring, condensed, diffuse, toroidal); chromosome size; number of chromosomes per cell; chromosome position (e.g., relative to cell boundaries); peptidoglycan synthesis (membrane formation); number of replication forks per cell; proton motive force (PMF); average distance between cell foci; channel formation/cell permeability; transcription ability; translation ability (accuracy or elongation); nucleotide synthesis ability; and position or intensity of a detectably labeled polynucleotide or polypeptide. Examples of proteins that can be tagged and detected are those involved in DNA replication (e.g., SeqA, DnaB), cell division (e.g., FtsZ, FtsA, FtsW), stress resistance (e.g., DegP, GroEL, Heat shock proteins), as well as bacterial actins (e.g., MreB, Alp7A, AlfA), and bacterial tubulins (e.g., FtsZ, TubZ).

In some embodiments, the at least one cytological characteristic is PMF and the control antibiotic with the known MOA is selected from CCCP, Azide, and SDP. In some embodiments, the at least one cytological characteristic is channel formation and the control antibiotic with the known MOA is selected from nisin and spirohexenolide A. In some embodiments, the at least one cytological characteristic is PG synthesis and/or chromosome shape, and the control antibiotic with the known MOA is selected from vancomycin, penicillin, and cephalexin. In some embodiments, the at least one cytological characteristic is chromosome position and/or cell size and the control antibiotic with the known MOA is selected from ciprofloxin and nalidixic acid. In some embodiments, the at least one cytological characteristic is chromosome shape and the control antibiotic is bleach. In some embodiments, the at least one cytological characteristic is chromosome shape and/or cell size and the control antibiotic is selected from rifampicin, tetracycline, choloramphenicol, trimethoprim, and kanamycin. In some embodiments, the at least one cytological characteristic is cell permeability and/or cell separation and the control antibiotic is chir-090.

One of skill will recognize that any number of cytological characteristics can be detected in any combination. In some embodiments, at least 2, 3, 4, 5, 6, 7, or 8 cytological characteristics are detected for any given antibiotic agent. In some embodiments, the cytological characteristic is detected using fluorescence microscopy, e.g., high throughput fluorescence microscopy.

In some embodiments, the method further comprises contacting the antibiotic agent with a microorganism (e.g., bacteria) of a different species or strain and detecting the effect of the antibiotic agent on the at least one cytological characteristic.

The present methods can be carried out on an individual cell basis, thus requiring smaller sample volumes, fewer cells, and smaller amounts of antibiotic agent (known or candidate agent) than previous methods. In some embodiments, the detecting is carried out with a small number of cells, e.g., less than 10⁵ cells per sample (less than 10, 100, 1000, or 10⁴ cells per sample). In some embodiments, the contacting is carried out with a small amount of antibiotic agent, at a concentration at or above the minimum inhibitory concentration (MIC) but in a small volume, e.g., less than 1 mL (e.g. less than 10, 50, 100, or 200 μL). Because of the sensitivity of the present methods for detecting the effect of antibiotic agents on bacteria, in some embodiments, the contacting is carried out with a concentration below the MIC of the antibiotic agent (e.g., 50, 60, 70, 80, 90, 95 or higher percent of the MIC).

Also provided are methods of selecting an antibiotic agent, e.g., from a pool of candidate antibiotic agents. The candidates can be analogs or modified versions of a known antibiotic agent, or new compounds from an uncharacterized sample or from a library of compounds. In some embodiments the method comprises contacting a microorganism with a candidate antibiotic agent, detecting at least one cytological characteristic of the contacted microorganism, comparing the effect of the candidate antibiotic agent on the at least one cytological characteristic with that of a control (or more than one control), wherein the control is the microorganism contacted with a known amount of an antibiotic agent with a known MOA, and selecting the candidate antibiotic agent if the same or lesser amount of the candidate antibiotic agent (compared to the known amount of the antibiotic agent with the known MOA) results in a greater effect on the at least one cytological characteristic than the known amount of the antibiotic agent with the known MOA. In some embodiments, the microorganism is bacteria (e.g., gram positive or gram negative).

In some embodiments, the candidate antibiotic agent and control antibiotic agent are contacted with the same strain or species of microorganism. In some embodiments, the method further comprises a negative control comprising uncontacted microorganism (e.g., the same microorganism not contacted with the candidate or control antibiotic agent). In some embodiments, the control represents an average value for the cytological characteristic of a plurality of antibiotic agents with the same known MOA, or an average value for the cytological characteristic obtained from more than one species or strain of microorganism. In some embodiments, the control is the microorganism separately contacted with a panel of antibiotic agents with different, known MOAs.

Again, any number of cytological characteristics, as listed above, can be detected in any combination. In some embodiments, at least 2, 3, 4, 5, 6, 7, or 8 cytological characteristics are detected for any given candidate antibiotic agent. In some embodiments, the cytological characteristic is detected using fluorescence microscopy, e.g., high throughput fluorescence microscopy.

In some embodiments, the method further comprises contacting the candidate antibiotic agent with a microorganism (e.g., bacteria) of a different species or strain and detecting the effect of the antibiotic agent on the at least one cytological characteristic. In some embodiments, the control antibiotic agent is also contacted with the different species or strain of microorganism.

As explained above, the present method can be carried out on an individual cell basis, thus requiring smaller sample volumes, fewer cells, and smaller amounts of antibiotic agent (known or candidate agent) than previous methods. In some embodiments, the detecting is carried out with a small number of cells, e.g., less than 10⁵ cells per sample (less than 10, 100, 1000, or 10⁴ cells per sample). In some embodiments, the contacting is carried out with a small amount of antibiotic agent, at a concentration at or above the minimum inhibitory concentration (MIC) but in a small volume, e.g., less than 1 mL (e.g. less than 10, 50, 100, or 200 μL).

Further provided are methods for classifying an antibiotic agent comprising contacting the antibiotic agent with a microorganism; detecting the effect of the antibiotic agent on at least one cytological characteristic (e.g., as listed above); comparing the effect of the antibiotic agent on the at least one cytological characteristic to that of a control (or multiple controls); and classifying the antibiotic agent. In some embodiments, the microorganism is bacteria (e.g., gram positive or gram negative). In some embodiments, the control is the microorganism (the same or different species or strain of the microorganism) not contacted with the antibiotic agent. In some embodiments, the control is the microorganism (the same or different species or strain of the microorganism) contacted with an antibiotic agent with a known MOA. In some embodiments, the control represents an average value for the cytological characteristic of a plurality of antibiotic agents with a known MOA, or an average value for the cytological characteristic obtained from more than one species or strain of microorganism.

In some embodiments, the method is carried out using a high throughput format (e.g., in a multiwell plate or a multispot surface), so that multiple antibiotic agents (or candidate antibiotic agents) can be classified at one time. In some embodiments, the control is the microorganism separately contacted with a panel of antibiotic agents with different, known MOAs.

Again, any number of cytological characteristics, as listed above, can be detected in any combination. In some embodiments, at least 2, 3, 4, 5, 6, 7, or 8 cytological characteristics are detected for any given candidate antibiotic agent. In some embodiments, the cytological characteristic is detected using fluorescence microscopy, e.g., high throughput fluorescence microscopy.

In some embodiments, the detecting is carried out with a small number of cells, e.g., less than 10⁵ cells per sample or well (less than 10, 100, 1000, or 10⁴ cells per sample or well). In some embodiments, the contacting is carried out with a small amount of antibiotic agent, at a concentration at or above the minimum inhibitory concentration (MIC) but in a small volume, e.g., less than 1 mL (e.g. less than 10, 50, 100, or 200 μL). In some embodiments, the contacting is carried out with a concentration below the MIC of the antibiotic agent (e.g., 50, 60, 70, 80, 90, 95 or higher percent of the MIC).

Provided herein are methods for characterizing the response of a bacterial strain to an antibacterial agent comprising contacting bacteria from the bacterial strain to the antibiotic agent; detecting the effect of the antibiotic agent on at least one cytological characteristic of the bacteria; comparing the effect of the antibiotic agent on the at least one cytological characteristic to a control (or more than one control), thereby characterizing the response of the bacterial strain to the antibacterial agent.

In some embodiments, the control is the bacteria (the same or different species or strain of bacteria) not contacted with the antibiotic agent. In some embodiments, the control is the bacteria (the same or different species or strain of bacteria) contacted with an antibiotic agent with a known MOA. In some embodiments, the control represents an average value for the cytological characteristic of a plurality of antibiotic agents with a known MOA, or an average value for the cytological characteristic obtained from more than one species or strain of bacteria. In some embodiments, the control is represented by the bacteria separately contacted with a panel of antibiotic agents with different, known MOAs. In some embodiments, the bacterial strain is resistant to at least one antibacterial agent.

In some embodiments, the at least one cytological characteristic is selected from the list provided above. In some embodiments, the at least one cytological characteristic and control antibiotic are described as above. Any number of cytological characteristics can be detected in any combination. In some embodiments, at least 2, 3, 4, 5, 6, 7, 8, 9, 10, or more cytological characteristics are detected for any given antibiotic agent. In some embodiments, the cytological characteristic is detected using fluorescence microscopy, e.g., high throughput fluorescence microscopy.

In some embodiments, the method further comprises contacting the antibiotic agent with bacteria of a different species or strain and detecting the effect of the antibiotic agent on the at least one cytological characteristic.

In some embodiments, the detecting is carried out with a small number of cells, e.g., less than 10⁵ cells per sample (less than 10, 100, 1000, or 10⁴ cells per sample). In some embodiments, the contacting is carried out with a small amount of antibiotic agent, at a concentration at or above the minimum inhibitory concentration (MIC) but in a small volume, e.g., less than 1 mL (e.g. less than 10, 50, 100, or 200 μL). In some embodiments, the contacting is carried out with a concentration of antibiotic agent that is below the MIC for the antibiotic agent (e.g., 50, 60, 70, 80, 90, 95 or higher percent of the MIC).

Further provided are methods of determining a course of treatment for an individual, surface, facility, food or water source, or eukaryotic cell culture infected by a bacterial strain by determining the antibiotic susceptibility of the bacterial strain. In some embodiments, the method comprises contacting bacteria from the bacterial strain with a panel comprising a plurality of antibiotic agents; detecting the effect of at least one antibiotic agent in the panel on at least one cytological characteristic (e.g., as listed above) of the bacteria; comparing the effect of the at least one antibiotic agent on the at least one cytological characteristic to that of a control (or more than one control), thereby determining the antibiotic susceptibility of the bacterial strain. In some embodiments, the effects of a plurality of antibiotic agents from the panel are detected. In some embodiments, the MOA of at least one antibiotic agent in the panel is known. In some embodiments, the MOA of all of the antibiotic agents in the panel are known. The course of treatment (e.g., for the individual, surface, facility, food or water source, or eukaryotic cell culture) can be determined by selecting the antibiotic agent that is most effective (e.g., kills rapidly, efficiently, or using a desired MOA) against the infecting bacterial strain. In some embodiments, at least 2, 3, 4, 5, 6, 7, 8, 9, 10, or more cytological characteristics are detected for any given antibiotic agent.

In some embodiments, the control is the bacteria (the same or different species or strain of bacteria) not contacted with the antibiotic agent. In some embodiments, the control is a different species or strain of bacteria contacted with the panel of antibiotic agents, wherein the same at least one cytological characteristic is compared to that of the infecting bacteria. In some embodiments, the control represents an average value for the cytological characteristic of a plurality of antibiotic agents with a known MOA, or an average value for the cytological characteristic obtained from more than one species or strain of bacteria.

Further provided are methods for creating a cytological profile of a bacterial strain, and using the profile to identify unknown bacteria, e.g., in a clinical sample, a surface, a facility, a food or water source, etc. In some embodiments, the method for generating a cytological profile comprises: (i) contacting bacteria from the bacterial strain with an antibiotic agent; (ii) detecting the effect of the antibiotic agent on at least one cytological characteristic of the bacteria (e.g., any cytological characteristic described herein); and (iii) comparing the effect of the antibiotic agent on the at least one cytological characteristic to that of a control (or more than one control), thereby creating a cytological profile of the bacterial strain. In some embodiments, the method further comprises repeating the steps (e.g., steps (i)-(iii)) for additional antibiotic agents, e.g., a second, third, forth, fifth, or more antibiotic agents. In some embodiments, the antibiotic agents have different MOAs (at least one antibiotic agent has a different MOA than the other(s)). In some embodiments, the control is uncontacted bacteria. In some embodiments, the bacteria contacted with the antibiotic agent and the control are the same bacterial strain. In some embodiments, the bacteria contacted with the antibiotic agent and the control are different bacterial strains. In some embodiments, at least 2, 3, 4, 5, 6, 7, 8, 9, 10, or more cytological characteristics are detected for any given antibiotic agent.

Provided herein are methods of using a cytological profile to identify bacteria. The bacteria can be obtained, e.g., from a clinical sample, a surface, a facility, a food or water source, etc. In some embodiments, the method comprises identifying bacteria from a sample by detecting at least one cytological characteristic of the bacteria (e.g., any cytological characteristic described herein); and comparing the at least one cytological characteristic of the bacteria to the cytological profile of at least one bacterial strain (e.g., a known or control bacterial strain), wherein the cytological profile indicates whether the bacteria belongs to the bacterial strain, thereby identifying bacteria from the sample. In some embodiments, the method comprises identifying bacteria from a sample by contacting the bacteria with an antibiotic agent; detecting the effect of the antibiotic agent on at least one cytological characteristic of the bacteria (e.g., any cytological characteristic described herein); and comparing the effect of the antibiotic agent on the bacteria to the cytological profile of at least one bacterial strain (e.g., a known or control bacterial strain), wherein the cytological profile indicates whether the bacteria belongs to the bacterial strain, thereby identifying bacteria from the sample. In some embodiments, the effect of the antibiotic agent on the bacteria is compared to the cytological profiles of two or more bacterial strains.

Further provided are methods for identifying and/or selecting an inhibitor of a eukaryotic protein by observing the effect of a candidate inhibitor on the cytological profile of bacteria expressing the eukaryotic enzyme. In some embodiments, the method comprises expressing a heterologous eukaryotic protein in a bacteria (e.g., using standard recombinant techniques); contacting the bacteria with a candidate inhibitor; detecting at least one cytological characteristic of the bacteria (e.g., any cytological characteristic described herein); comparing the effect of the candidate inhibitor on the at least one cytological characteristic to that of a control (or more than one control); selecting the candidate antibiotic agent if the candidate inhibitor has an effect on the at least one cytological characteristic. In some embodiments, the control is uncontacted bacteria. In some embodiments, the control is bacteria that do not express the heterologous eukaryotic protein. In some embodiments, the control is bacteria contacted with an antibiotic agent with a known MOA. In some embodiments, the bacteria expressing the heterologous eukaryotic protein and the control are the same bacterial strain. In some embodiments, the bacteria expressing the heterologous eukaryotic protein and the control are from different bacterial strains. In some embodiments, at least 2, 3, 4, 5, 6, 7, 8, 9, 10, or more cytological characteristics are detected for a given candidate inhibitor.

### BRIEF DESCRIPTION OF THE DRAWINGS

FIG. 1. Summary of cytological profiles for drugs with the indicated mechanisms of action (MOA). Untreated cells continue to grow and maintain normal cell size, permeability and structure. Cells treated with compounds that deplete cellular ATP or the proton motive force (PMF) show decondensed chromosomes at early times. Later, Gram-positive B. subtilis cells show dramatic cell lysis caused by autolytic enzymes degrading the wall of cells with high internal osmotic pressure. Cells treated with compounds that form large channels (e.g., Nisin) show increased permeability at early times. Later, Gram-positive B. subtilis cells show autolysis, but the cells have low internal osmotic pressure and less dramatic lysis. Sequestration of the Lipid II peptidoglycan precursor (e.g., with vancomycin) produces large internal membrane vesicles, while inhibition of peptidoglycan crosslinking (e.g. with penicillin) causes chromosome condensation and lysis. Inhibition of septum-specific peptidoglycan biosynthesis by cephalexin produces elongated cells with multiple chromosomes. Inhibition of chromosome segregation by DNA gyrase inhibitors (e.g., ciprofloxin, nalidixic acid) produces elongated cells with centrally localized and fragmented DNA. Oxidative damage by bleach causes the chromosome to condense into a dramatic ribbon-like structure, while the inhibition of transcription (e.g., with rifampicin) causes the chromosome to decondense and produce wider cells. Inhibition of translational elongation (e.g., with tetracycline or chloramphenicol) leads to a toroidal chromosome and wider cells. Reduced accuracy of translation (e.g., with kanamycin) produces wider cells, with chromosomes condensed into a non-toroidal structure. Nucleotide biogenesis inhibitors (e.g. trimethoprim) condense the chromosome without affecting cell width. Inhibiting synthesis of the LPS component of the Gram-negative outer membrane (e.g., with CHIR-090) produces chains of cells with increased permeability. Compounds listed in italics are shown herein to have the indicated cytological profile (see e.g., FIG. 8).

FIG. 2. An example of E. coli cells treated with two different antibiotics. The E. coli strain contains a biomarker (SeqA-GFP) as a reporter of the position of DNA replication forks. Cells were grown in LB media at 30° C. and then were either untreated, or treated with nalidixic acid or cephalexin. Cell length, cell width, chromosome morphology (length and width), the number of chromosomes per cell, the position of the chromosomes, number of SeqA-GFP foci per cell, the position of the foci with respect the chromosome, average distance between foci, and intensity of foci were measured, and variance determined for each. Cells treated with cephalexin or nalidixic acid have a cytological profile that is dramatically different from the untreated control and from each other. Specifically, the untreated cells have chromosomes that span ˜85% of the length of the cell, with 1-4 SeqA foci per chromosome, cephalexin treated cells contain multiple SeqA-GFP foci that are spread uniformly throughout the cell, while naladixic acid treated cells contain highly elongated chromosomes in which the SeqA foci are clustered together

FIG. 3. Cell length distributions for wild type untreated cells (green) compared to cells treated with either a lethal dose (black) or sublethal dose (red) of nalidixic acid. Cells were prepared as for FIG. 2 and cell lengths were measured. These measurements were grouped into bin sizes of 1 micron and plotted versus the number of cells in each bin. A total of 50 cells were measured for each condition. Sublethal concentrations of nalidixic acid caused elongation.

FIG. 4. Chromosome length distribution for a population of wild type cells (green bars) is compared to cells treated with two different concentrations of (A) nalidixic acid or (B) chloramphenicol for 2 hours. (A) NaI 0.5 (blue bars on the left; 1×MIC), cells treated with nalidixic acid at its MIC (0.5 μg/ml) under these conditions. NaI 0.1 (red bars in the middle; ⅕×MIC), cells treated with nalidixic acid at 5 fold below its MIC (0.1 μg/ml). Untreated cells are indicated in the green (right) bars. (B) Cam 5 (red bars on the right), cells treated with chloramphenicol at its MIC (5 μg/ml). Cam I (blue bars in the middle), cells treated with chloramphenicol at 5 fold below its MIC (1 μg/ml). Green bars on the left indicate results for untreated cells. Chromosome lengths were measured and grouped into bin sizes of 0.5 microns and plotted versus the number of cells in each bin. A total of 50 cells were measured for each condition.

FIG. 5. Differential effect of protein synthesis inhibitors at MIC on E. coli chromosome morphology. Cells were grown for 2 hours in the presence of chloramphenicol (5 μg/ml), tetracycline (20 μg/ml), nalidixic acid, kanamycin (50 μg/ml) or streptomycin (50 μg/ml) and then stained with the membrane stain FM 4-64 (red) and DAPI (blue) and imaged with a fluorescence microscope. The percentage of cells (n=50) in which the chromosome condensed to form a ring was quantified for each growth condition. Representative images for wild type untreated cells, or cells grown in the presence of chloramphenicol, tetracycline, and streptomycin are shown. Streptomycin and kanamycin treated cells have a similar appearance. Similar effects were seen in B. subtilis.

FIG. 6. The distance between the chromosome border and the cell pole after treatment with chloramphenicol. Distribution of the distance between chromosomes and cell poles for a population of wild type cells (green, right bar) is compared to cells treated for two hours with two different concentrations of chloramphenicol. Cam 1 (red, middle bar) represents the data for cells treated with chloramphenicol below the MIC (1 μg/ml). Cam 5 (black, left bar) represents the data for cells treated with chloramphenicol at the MIC (5 μg/ml). Distances were measured and grouped into bin sizes of 0.5 microns and plotted versus the number of cells in each bin. A total of 100 measurements were made for each condition.

FIG. 7. Cytological profiling of E. coli cells (A) Untreated, or treated with Tetracycline (B. Tet), Chloramphenicol (C. Cam), Rifampicin (D. Rif) and Naladixic Acid (E. NaI) for two hr at ⅕× the MIC, with NaI also shown at 1× the MIC (F). FIG. 7G shows the growth curve, with red arrow indicating time at which images were collected. Cells treated with NaI at ⅕× the MIC show normal growth, but abnormal morphology.

FIG. 8. Affect of various treatments on Bacillus subtilis cell architecture and permeability. (A-J) Cytological profiling. Cells were stained with the membrane stain FM 4-64 (red) and the membrane impermeable nucleotide stains Sytox Green (green) and DAPI (blue) and visualized 2 hours after treatment unless otherwise noted. (A) Untreated cells show uniform FM 4-64 staining, faint DAPI staining and no Sytox green staining. (B) Nisin treated cells rapidly become permeable to DAPI and Sytox Green and have uneven membrane staining. Cells imaged after 1 hr of treatment. (C) Vancomycin treated cells show internal membrane spheres and blebs. (D) Penicillin G treated cells show condensed chromosomes, and some cells show increased permeability to Sytox and DAPI and gaps in the cytoplasmic membrane. (E) Azide and (F) CCCP treated cells show some cells with large gaps in the cytoplasmic membrane and increased permeability to Sytox and DAPI as well as membrane debris and vesicles. (G) SDP treated cells appear similar to Azide and CCCP treated cells, and to cells that were not aerated (H). The dramatic lysis of SDP (I) and statically incubated cultures (J) depends on the autolysis enzymes encoded by lytA, lytB, lytC, and lytD. Cells were imaged after 8 hours. (K) Cell permeability as assessed by Sytox staining intensity per pixel. Nisin shows increased permeability. (L) The DAPI staining intensity/pixel reflects increased permeability (seen here only for Nisin) and increased chromosome condensation. Both SDP and CCCP treated cells have decondensed chromosomes (also visible in F-G), while Vancomycin and Nisin cause more condensed chromosomes (also visible in B-C). (M) Cell viability decreases within 10 minutes of SDP treatment, with lysis occurring within hours. (N) The lysis defective strain (ALBI 111) also shows rapid decreases in viability, but does not lyse. (O) SDP and Nisin rapidly collapse the proton motive force (PMF) and depolarize the membranes, while Vancomycin at the MIC has no effect on PMF, as determined by fluorometer assay.

FIG. 9. Antibiotics with different MOA fall into different, clearly defined categories. (A) Quantitative analysis of cellular parameters can separate antibiotics by mechanism of action. Values for the y-axis were generated by dividing the average distance between the chromosome and the pole of the cell by the average cell length. E. coli were treated with the indicated antibiotic at the MIC for two hours and then imaged using a fluorescence microscope after staining the cell membranes and the DNA. Measurements were made for 50 cells for each condition. Rif: rifampicin; Tmp: trimethoprim; Kan; kanamycin; Tet: tetracycline; Cam: chloramphenicol; Cip: ciprofloxacin; NaI: naladixic acid; NaOCl: bleach. (B) Principal component analysis (PCA) shows that the two principle components plotted account for 85% of the variability.

FIG. 10. Measurements of DAPI staining for single cells after 10 minute exposure to nisin, Spirohexenolide A, chlorothricin, and DMSO. The graph shows fluorescence intensity of DAPI staining corresponding to a line drawn through the middle of each of the cells shown at the bottom. Cells treated with nisin, SpiroA, and chlorothricin show increased fluorescence comparted to untreated control.

FIG. 11. Cytological profiling of polymyxin B treated cells. The data show that Polymyxin B inhibits outer membrane biogenesis similar to Chir-090. Images show an example of cytological profiling of E. coli after two hours of treatment at the MIC with Chir-090 (left) and polymyxin B (right).

FIG. 12. Identification of antibiotic activity in B. subtilis strain 3610. (A) The agar and the bacteria were extracted from solid culture with 95% methanol to remove potential natural products. The methanol extract was fractioned on a C18 SepPak column, and eluted with solvents of increasing hydrophobicity. The solvents were removed using a speed vac and the samples were resuspended in water. Each fraction was screened for antibiotic activity by spotting the extracts onto a lawn of E. coli and by cytological profiling. The 80% Methanol fraction contained compounds that kill E. coli, producing a zone of clearing. (B) Cytological profiling showing that the same B. subtilis crude extract contains a compound that causes the toroidal chromosomes produced by treatment with translational inhibitors such as Tet and Cam. A comparable crude extract from a pks mutant that does not produce bacillaene has no effect on the cells, suggesting that bacillaene is the compound responsible for the cytological effects. (C) The crude fraction was separated using an HPLC column and fractions were subject to mass spectrometry electrospray ionization. Molecular ions with masses corresponding to bacillaene A and B were detected, as were the Na salt of these ions.

### DETAILED DESCRIPTION OF THE INVENTION

### I. Introduction

Provided herein are methods and assays for rapidly identifying new antibiotics and determining their mechanisms of action (MOAs). These approaches can also be used in a clinical setting for diagnostic testing of microorganisms, e.g., for antibiotic susceptibility.

Different classes of antibiotics have different cytological effects on cells, resulting in a cytological profile that is characteristic for each class of antibiotic. This phenomenon is referred to herein as multifactorial imaging cytological profilng. The present methods can be used to screen for antibiotics that affect any bacterial species by any mechanism of action, known or unknown. The present methods can rapidly distinguish between known classes of antibiotics, thereby allowing for rapid determination of MOAs for novel compounds. The present methods also provide insight into the mechanism of antibiotic resistance when it arises. The present methods can be used to screen for compounds (e.g. derivatives of known antibiotics) that have lower minimal inhibitory concentrations (MICs), different or improved killing kinetics, different mechanisms of action, as well as those that affect different subpopulations of cells or in different conditions such as in a biofilm.

The approach described below uses high throughput microscopy and multifactorial image analysis to identify compounds that adversely affect microbial cells. One of skill will appreciate that other techniques can be used, or may be developed, to detect particular cytological parameters, e.g., flow cytometry for fluorescence-based determinations. Cytological profiling as described herein measures the cytological effects of known or candidate antibiotic compounds on bacteria. The method monitors these effects on individual cells, and is thus extremely sensitive, detecting effects at much lower concentrations than can be achieved in population based assays. The single cell approach, using microscopy as described in the examples, has many advantages over population based compound screening approaches:
1. The approach is a single cell assay and can detect effects on subpopulations of cells.
2. The approach is highly sensitive and can detect adverse effects on bacterial cells well below the MIC and at concentrations of compound where cell growth is not inhibited using standard growth assays.
3. A very small number of cells are needed, so it can be performed in a high density microscale format. Thus, only a very small amount of the test and control agents are necessary. This is advantageous in clinical situations, where a small number of bacteria obtained from a patient can be used to determine a course of treatment and determine if the treatment is effective over time.
4. This approach can be used to screen for effects on biotargets (proteins) that may be present at very low levels within the cell, such as essential cell cycle or cell division proteins.
5. The comprehensive cytological analysis can reveal unexpected effects of drugs on cells, such as the ability of protein synthesis inhibitors to cause dramatic reorganization of the E. coli chromosome into a donut shape.
6. The approach is very rapid, providing a readout on antibiotic susceptibility and compound effects within a few hours, instead of an entire day for traditional antibiotic sensitivity tests.
7. The approach can be broadly applied to identify antibiotics that affect any microorganism, including fastidious bacteria like Mycobacterium tuberculosis.
8. This versatile approach can be used to screen compounds for activity when cells are not rapidly growing, such as in a biofilm, an abcess, or in stationary phase, conditions where growth dependent assays fail. The advantage of this approach is that it does not depend on measuring cell growth over time like most other assays, but instead monitors cellular responses over time on a single cell basis. For example, one can screen antibiotics and derivatives thereof that lead to bacterial death in more complex conditions, such as in a biofilm or in the presence of surfactant.
9. The approach immediately provides mechanistic information about the mode of action.
10. The approach provides information about the kinetics of cell death and whether a given compound is bacteriostatic or bacteriocidal.
11. The approach distinguishes agents based on MOA, and can thus be used to screen for compounds that affect different cellular processes, e.g., to screen libraries for compounds with modified target specificity.
12. The approach of using a combination of fluorescent stains, protein-GFP fusions, and multiple cytological measurements to investigate antibiotic activity has never been used with bacteria under conditions capable of detecting subtle changes in cytological characteristics and distinguishing antibiotics with different MOAs.
13. The method provides a graphic view of cell death in response to antibiotic action, which can hasten understanding and acceptance of new antibiotics by commercial and regulatory communities.
14. The methods can be used to identify bacteria, e.g., in clinical setting. Based on cytological profiles developed for individual bacterial strains in the presence of various antibiotic agents, unknown bacteria can be distinguished, and often specifically identified, as belonging to a particular strain.
15. The present approach can be used to screen for inhibitors of eukaryotic enzymes. The eukaryotic enzyme can be one that corresponds to a known bacterial homolog. By targeting the bacterial homolog with a candidate agent and determining the effect of agent on the cytological profile of the bacteria, drugs can be developed to target the eukaryotic enzyme in a desired manner. Alternatively, the eukaryotic enzyme can be expressed in bacteria, and targeted with a candidate agent. The cytological characteristics of the bacteria in the presence of the agent can be observed, and an agent with the desired effect can be selected. Such methods can be used to identify agents to target cancer, malaria, pathogenic fungus, etc.

### II. Definitions

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

An “antibiotic agent” or “antimicrobial agent” refers to a compound that has the ability to reduce the growth or viability of a microorganism. An antibiotic agent can be composed of either organic or inorganic compounds, and can include bioactive molecules produced by the immune system. Generally, the terms are used interchangeably with “antibacterial agent,” as bacteria are the most commonly targeted microorganisms. The cell-killing activity of an antibiotic agent is commonly determined in relative terms, e.g., in comparison to a control (such as untreated cells or cells treated with a known antibiotic), but can be expressed in quantitative terms as well (e.g., cell killing units/ng agent). Commonly, antibiotic activity will be initially observed for a given agent as a general phenomenon, e.g., with reduced cell growth or viability. The agent is then typically referred to as an antibiotic, even if the MOA or the quantitative efficacy is not known.

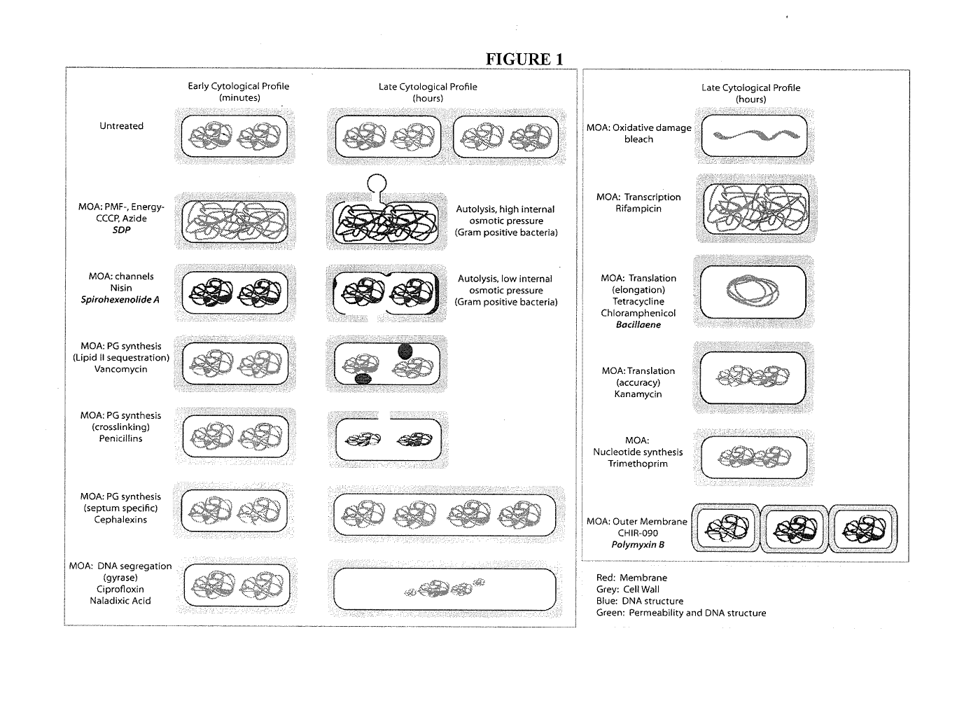

MOA refers to the “mode” or “mechanism of action.” An antibiotic can have more than one MOA. One of skill will recognize that an MOA that affects a certain pathway, e.g., cell division, can be described in different ways, e.g., identified by different molecular members of the same pathway. Examples of MOAs and corresponding antibiotic agents are shown in FIG. 1 and described in the Examples.

A cytological characteristic is an observable (quantifiable or comparable) cell feature. Examples include cell size (e.g., length, width, volume); chromosome (or nucleoid) shape (length and width), number, and position; amount and localization of a detectable protein (e.g., a protein associated with the foci or chromosome), the number of replication forks per cell, the proton motive force (PMF), etc. Examples of proteins that can be tagged and detected are those involved in DNA replication (e.g., SeqA, DnaB), cell division (e.g., FtsZ, FtsA, FtsW), stress resistance (e.g., DegP, GroEL, heat shock proteins), as well as bacterial actins (e.g., MreB, Alp7A, AlfA), and bacterial tubulins (e.g., FtsZ, TubZ).

The term “cytological profile” refers to the cytological characteristics observed in a particular bacterial strain under various conditions, e.g., in the presence or absence of an antibiotic agent, or in the presence of different antibiotic agents. Similarly, a particular antibiotic agent can have a cytological profile, comprising the cytological characteristics that it causes in various bacteria.

The terms “agonist,” “activator,” “inducer” and like terms refer to molecules that increase activity or expression as compared to a control (e.g., a negative control). Agonists are agents that, e.g., bind to, stimulate, increase, activate, enhance activation, sensitize or upregulate the activity of the target. The activity can be increased 10%, 20%, 30%, 40%, 50%, 60%, 70%, 80%, 90% 100% or more than that in a control. In certain instances, the activation is 1.5-fold, 2-fold, 3-fold, 4-fold, 5-fold, 10-fold, or more in comparison to a control.

The terms “inhibitor,” “repressor” or “antagonist” or “downregulator” interchangeably refer to a substance that results in a detectably lower activity level as compared to a control (e.g., a negative control). The inhibited activity can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. Thus, the control need not represent a side-by-side comparison, but a comparison to an average or value obtained at a different time. One of skill in the art will recognize that controls can be designed for assessment of any number of parameters.

For example, a control can be devised to compare qualitative (e.g., comparative brightness) or quantitative measures (e.g., objective length), or therapeutic measures (e.g., comparison of likely or actual benefit and/or side effects). Controls can be designed for in vitro or in vivo applications. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or proteins or other entities which can be made detectable, e.g., by incorporating a radiolabel into a peptide, antibiotic, or antibody specifically reactive with a target. Any method known in the art for conjugating an appropriate molecule to the label may be employed, e.g., using methods described in Hermanson, Bioconjugate Techniques 1996, Academic Press, Inc., San Diego.

A “labeled” molecule (e.g., nucleic acid, protein, or antibody) is one that is bound, either covalently, through a linker or a chemical bond, or noncovalently, through ionic, van der Waals, electrostatic, or hydrogen bonds to a label such that the presence of the molecule may be detected by detecting the presence of the label bound to the molecule.

A cell is considered “viable” if it is alive and capable of growth. The number of viable bacterial cells in a given sample can be determined directly, e.g., using a microscope, or using plate counts at various dilutions. Roszak and Colwell (1987) Applied Environ. Microbiol. 53:2889 describe an additional method for determining viability based on incorporation of radiolabeled substrates.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA.

The terms “identical” or “percent identity,” in the context of two or more nucleic acids, or two or more proteins, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 15, 20, 25, 30, 35, 40, 45 or more amino acids or nucleotides in length, e.g., 25-40, 30-40, 30-45, 40-80, or over a region that is 50-100 amino acids or nucleotides in length. In some cases, the percent identity of corresponding (e.g., homologous) sequences is determined over the length of a functional domain, more than one functional domain, or over the entire length of the sequence. The percent identity of corresponding functional domains (and sequences encoding functional domains) is typically higher than that of intervening sequences.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid can be used interchangeably with gene, cDNA, mRNA, oligonucleotide, and polynucleotide.

The words “protein,” “peptide,” and “polypeptide” refer an amino acid polymer or a set of two or more interacting or bound amino acid polymers. The terms can also apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or substantially identical amino acid sequences, or where the nucleic acid does not encode an amino acid sequence, to substantially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Substitution of a given residue with a different amino acid that does not result in a substantial change in the activity of the protein can indicate that the substitution is conservative.

The following groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and animals are those that express a heterologous gene or coding sequence, typically as a result of recombinant methods.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein). An example of a heterologous protein is a GFP protein (i.e., a GFP protein sequence combined with a different protein sequence to form a single polypeptide chain).

The terms “culture,” “culturing,” “grow,” “growing,” “maintain,” “maintaining,” “expand,” “expanding,” etc., when referring to cell culture itself or the process of culturing, can be used interchangeably to mean that a cell is maintained outside the body (e.g., ex vivo) under conditions suitable for survival. Cultured cells are allowed to survive, and culturing can result in cell growth, differentiation, or division. The term does not imply that all cells in the culture survive or grow or divide, as some may naturally senesce, etc. Cells are typically cultured in media, which can be changed during the course of the culture.

The terms “media” and “culture solution” refer to the cell culture milieu. Media is typically an isotonic solution, and can be liquid, gelatinous, or semi-solid, e.g., to provide a matrix for cell adhesion or support. Media, as used herein, can include the components for nutritional, chemical, and structural support necessary for culturing a cell.

### III. Methods for Detecting Cytological Characteristics

Cytological characteristics can be conveniently determined by fluorescence microscopy, which can be used for detection of qualitative (e.g. comparative staining profiles) and quantitative (e.g., cell length and other subcellular dimensions) characteristics. Other microscopic methods can also be used, such as light and electron microscopy. Microscopy is known in the art, and described in, e.g., Pogliano et al. (2001) PNAS 98:4486 and Liu et al. (2006) Mol. Microbiol. 59:1097.

For example, a culture can be harvested and resuspended in PBS, and an aliquot of the cell suspension placed on an agarose pad or slide. Optionally, the agarose pad can be heated on the microscope stage (e.g., using an environmental chamber). Images of cells are captured at one or more times. For time lapse experiments, cells can be deposited as described, and images captured at intervals (e.g., 30 second or 1 minute intervals). Images can be captured using an optical sectioning microscope, including a microscope, objective, camera, and fluorescence filters. Standard fluorescence filters include: FITC for GFP visualization (excitation, blue 490/20 nm; emission, green 528/38 nm) and RD-TR-PE for FM 4-64 and mCherryFP visualization (excitation, green 550/28 nm; emission, orange 617/73 nm). Software can be used to apply an algorithm to deconvolve the images, e.g., a constrained iterative deconvolution algorithm. Following deconvolution, the brightness and contrast of each fluorochrome can be adjusted to set the area outside of cells to be background.

Fluorescence microscopy can be modified for high throughput and/or real time measurements. Such methods are described in Fero & Pogliano (2010) Cold Spring Harbor Perspect Biol 2:a000455, which describes a quantitative data reduction approach.

Imaging mass spectrometry can also be used for detecting certain metabolic parameters, e.g., as described in Gonzalez et al. (2011) Microbiol. and Yang et al. (2009) Nat. Chem. Biol. 5:885. As an example, an agar sample containing bacteria can be placed on a MALDI target plate and subjected to Bruker microflex MALDI-TOF MS for imaging. The image is typically analyzed using software (e.g., FlexImaging 2.0 software).

Additional fluorescence based methods, such as flow cytometry and FACS, can be used for detecting certain labeled or stained cytological components. These data can indicate the intensity of staining, the presence and intensity of multiple stains/fluorophores, and other useful characteristics such as cell size and viability. Microfluidic devices that allow for precise control of miniaturized culture volumes can also be used. Nanofabricated devices can be used to sort bacteria from patient samples based on size, so that bacteria can be isolated for cytological testing.

One of skill in the art will recognize that detection techniques are constantly improving, so that new methods will be developed for detecting various cytological characteristics. Thus, while the present disclosure relies primarily on microscopy, any method for detecting cell size (e.g., length, width, volume); chromosome number, size, and shape; subcellular distances (e.g., between loci, or between loci and a given chromatin protein, etc.); and metabolic characteristics (e.g., PMF) can be used.

### IV. Microorganisms and Bacterial Strains

The small size of bacterial cells has hindered understanding of the cytological effects of small molecules and antibiotics. Achieving sufficient resolution to quantify changes in bacterial cell structure is challenging and requires high magnification. Furthermore, bacteria lack the cellular compartments that enabled this approach in eukaryotes (e.g., nucleus, golgi, mitochondria, vacuoles, secretory vesicles). In addition, bacterial cell division is not accompanied by the dramatic structural changes associated with eukaryotic mitosis, and bacteria lack many of the readily visualized proteins, including spindle pole bodies, microtubules, actins, motor proteins, etc. Therefore, little effort has been made to apply cytological profiling to study the impact of antimicrobial compounds on bacteria. As described in more detail below, high-resolution fluorescence microscopy can be used to assess cytological parameters and distinguish between different classes of antimicrobial agents, based on reproducible effects on the cell architecture (summarized in FIG. 1). The presently described cytological profiling assay can be used to screen for new anti-infective and antibiotic compounds, to assess the antibiotic sensitivity of clinical isolates, and to classify the activity of newly identified antimicrobial agents in crude extracts or after purification.

Examples of bacterial genera which can be used for the disclosed methods include Escherichia, Salmonella, Pseudomonas, Klebsiella, Acinetobacter, Anaplasma, Butyrivibrio, Rhodococcus, Bifidobacterium, Laribacter, Pantoea, Mycobacterium, Glossina, Helicobacter, Synechococcus, Synechocystis, Caulobacter, Streptomyces, Rickettsia, Campylobacter, Neisseria, Arcobacter, Streptococcus, Staphylococcus, Yersinia, Bordetella, Candidatus, Chlamydia, Borrelia, etc. In some embodiments, the bacteria are gram positive (e.g., Streptococcus, Staphylococcus, Clostridium, Listeria, Bacillus). In some embodiments, the bacteria are gram negative (e.g., Enterobacteriaceae, Neiseria, Vibrio, Campylobactor).

The present methods do not require observation of complex eukaryotic cell structures, and can be applied to other microorganisms as well. Aside from gram positive and gram negative bacteria, the present methods can be applied to, e.g., mycobacteria and mycoplasma.

### V. Antibiotic Agents

FIG. 1 sets out general classes of antibacterial agents, with examples of each class. Antibiotics have been developed and modified for generations, and several comprehensive reviews describe known antibiotic agents (see, e.g., Walsh, Antibiotics: Actions, Origins, Resistance (2003); Grayson, Kucer's Use of Antibiotics (2010)).

A brief list of antibiotics includes, but is not limited to
Carbacephems: Inhibit cell wall synthesis
Aminoglycosides: Inhibit translation by binding 30S ribosomal subunit
Macrolides and lincosamides: Inhibit translation by binding 50S ribosomal subunit
Daptomycin: Membrane disruption, disruption of PMF
Beta-lactams (including penicillins), cephalosporins, monobactams, and glycopeptides: Inhibit PG synthesis and disrupt bacterial cell wall
Quinolones: Inhibit DNA replication/transcription by inhibiting DNA gyrase
Sulfonamides: Inhibit nucleic acid synthesis by inhibiting folate synthesis
Tetracyclines: Inhibit translation through binding to 30S ribosomal subunit.

Representatives of these classes can be used to create a cytological profile for a given bacterial strain, to create a cytological profile for the class, e.g., in a variety of bacterial strains, or as controls for testing new or candidate antibiotic agents.

### VI. Kits

Further provided are kits for producing a cytological profile. In some embodiments, the kit includes at least one of the following components: a nucleic acid/chromatin stain (e.g., DAPI, Sytox green, acridine orange (AO), ethidium bromide (EB), haematoxylin, hoechst), a cell wall stain (e.g., crystal violet, gram stain, calcofluor, solophenyl flavine, pontamine fast scarlet), a membrane potential sensitive stain (e.g., 3,3′-diethyloxacarbocyanine iodide or DiOC₂, 3,3′-Dipropylthiadicarbocyanine iodide or DiSC₃) and a cell membrane stain (e.g., FM4-64, CTC). In some embodiments, the kit includes a nucleic acid stain and a cell wall stain. In some embodiments, the kit includes a cell membrane stain and a nucleic acid stain. In some embodiments, the kit comprises all listed components.

The kit can also include control antibiotic compounds with known MOAs. For example, the kit can include at least one of the following: protein synthesis inhibitors (e.g., chloramphenicol, streptomycin, tetracycline, or kanamycin), DNA gyrase inhibitors (e.g., nalidixic acid), cell wall inhibitors (e.g., vancomycin, cephalexin, or penicillin G), energetic poisons/PMF inhibitors (e.g., nisin, azide, or CCCP); immune system effectors (e.g., antibodies); and nonspecific inhibitors (e.g., bleach, ethidium bromide, SDS).

The kit can also include bacteria to be used for generating a cytological profile. The bacteria can be of any strain, and can be wild type or genetically modified. Useful genetically modified strains will include those that encode tagged proteins, e.g., fusion proteins that include a chromatin protein component or DNA binding protein component and a GFP or other detectable component. In some embodiments, the tagged protein is a fusion protein that includes a protein component that binds a bacterial cell structure and a GFP or other detectable component. An example is SeqA-GFP, which detects the hemimethylated DNA at replication foci. The genetically modified bacterial strain can also be one that is defective in a pathway or gene that affects the cytological profile, e.g., mutant strains with defective autolysis or phospholipid (cell membrane) synthesis. An exemplary kit includes representative of more than one bacterial strain, e.g., a gram negative and a gram positive strain.

The kit can also include consumable items, e.g., culture media or media stock solution or powder; culture labware, such as multiwall plates, tubes, pipettes; slides, e.g., for visualization, etc.

One of skill will understand that any number of these components can be included in the kit in any combination. For example, the kit can include stains for cytological components (e.g., nucleic acid, cell membrane, or cell wall) and at least one, two, three, four, or more antibiotic agents with known MOAs. In some embodiments, the kit further includes samples of at least one or two bacterial strains.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entireties for all purposes.

### VII. Examples

### A. Example 1

### Cytological Effects of Antimicrobials on Gram Positive and Gram Negative Bacteria

To determine if different classes of antimicrobials can be distinguished based on cytological parameters, comparative cytological analysis of Escherichia coli and Bacillus subtilis cells treated with several different classes of antibiotics was performed. The study included several different protein synthesis inhibitors (chloramphenicol, streptomycin, tetracycline, kanamycin), DNA gyrase inhibitors (nalidixic acid), cell wall inhibitors (vancomycin, cephalexin, penicillin G), and energetic poisons (nisin, azide, CCCP). Also included were nonspecific inhibitors such as bleach, ethidium bromide, and sodium docecyl sulfate. The results indicate that each antibiotic with a different mechanism of action (MOA) generates a unique cytological profile that is also different from the nonspecific inhibitors. This analysis is expanded to molecules with unknown MOAs as described below.

### B. Example 2

### Determination of MIC

The minimal inhibitory concentration (MIC) of each antibiotic was determined for E. coli strains JP750 or W3110, and for Bacillus subtilis strain PY79. A test tube containing 5 ml of LB was inoculated with a single colony and grown until the culture reached a density of approximately 2×10⁸ cells/ml. Small inoculums (20 μl) of this culture were then diluted into LB containing various concentrations of antibiotic ranging from 0 to 200 μg/ml and the cultures were incubated overnight at 30° C. The MIC was the lowest concentration that blocked growth.

### C. Example 3

### Cytological Profiling of E. Coli

The dose-dependent cytological profiles for the various classes of antibiotics was determined. In general, a series of cultures of the appropriate strain were grown in LB at 30° C. until the cultures reached a density of approximately 2×10⁸ cells/ml, at which point the compound of interest was added so that the final concentrations ranged from 0 to 200 μg/ml. For each compound, samples representing ⅕×, 1× and 5× the MIC were examined. Samples were taken at various times after addition of drug (ranging from 0.2 to 3 hours). The cells were stained with FM 4-64 (1 μg/ml) to visualize the cell membranes and DAPI (5 μg/ml) to visualize the chromosome (Pogliano et al. (1999) Mol. Microbiol. 31:1149). Samples can also be incubated in very small volumes (10 μl or less) with similar results.

Images of each stain and any fluorescently-tagged fusion protein present in the strain were then captured using a high resolution microscope. Cell length and width, chromosome length and shape, distance of the chromosome from each cell pole, the number of chromosomes per cell, and the presence and distribution of GFP (or GFP fusions) present within the cells were measured.

In the specific examples described below, E. coli strain JP750 was grown for 2 hours at 30° C. in the presence of various concentrations of nalidixic acid, chloramphenicol, or cephalexin, ranging from 0 to 30 μg/ml. Strain JP750 expresses a SeqA-GFP fusion from an IPTG dependent promoter. SeqA-GFP forms fluorescent foci that indicate the position of DNA replication forks/hemimethylated DNA within the cell (Brendler et al. (2000) EMBO 19:6249). After two hours of growth, samples were taken, stained with FM 4-64 (1 μg/ml) to visualize the cell membranes, and DAPI (5 μg/ml) to visualize the chromosome.

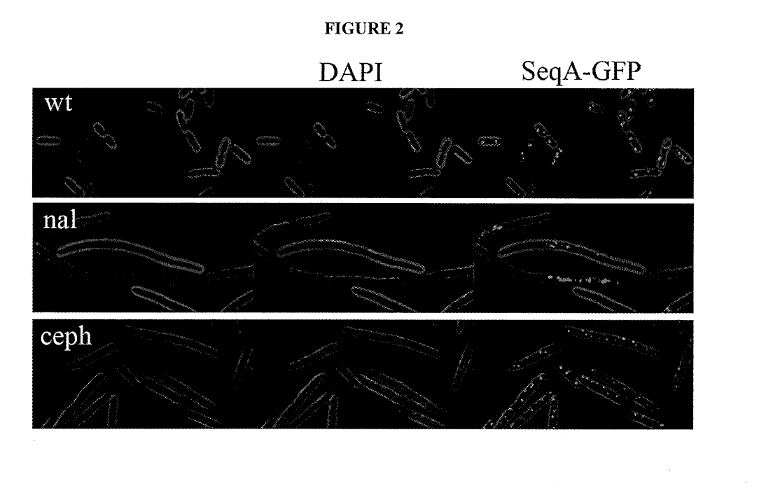

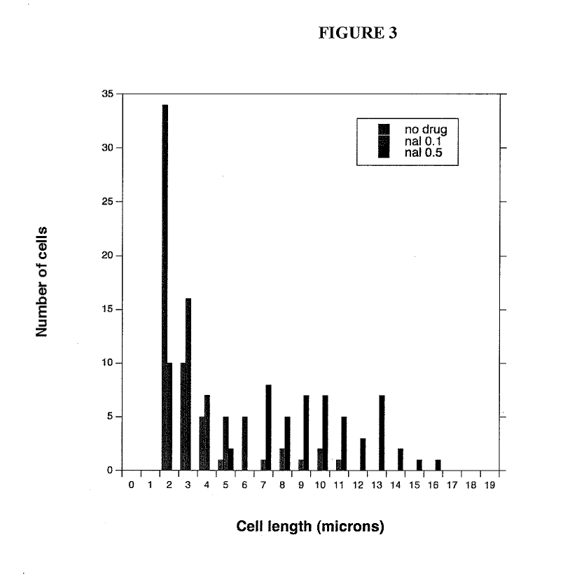

Cell length measurements were first obtained for each sample. FIG. 2 shows images of E. coli cells after treatment with cephalexin and nalidixic acid. FIG. 3 shows a series of histograms of the size length distribution for populations of cells grown in different concentrations of nalidixic acid. Untreated (wt) cells ranged in length from 1.8 to 4.2 microns. In contrast, cells treated with a lethal concentration of the gyrase inhibitor nalidixic acid (0.5 μg/ml) were elongated, ranging from 5 to 16 microns. It is known, however, that antibiotics with different MOAs can cause cell elongation. For example, cells treated with cephalexin, which binds to a penicillin binding protein required for cell wall biogenesis during cell division, are also elongated (FIG. 2) (Pogliano et al. (1997) PNAS 94:559; Drawz & Bonomo (2010) Clin. Microbiol. Rev. 23:160). While cell length alone did not distinguish between classes of antibiotics, the results shown below demonstrate that including other morphological features and/or fluorescently tagged fusion proteins allows various drug classes to be distinguished.

### D. Example 4

### Chromosome Structure after Nalidixic Acid and Chloramphenicol

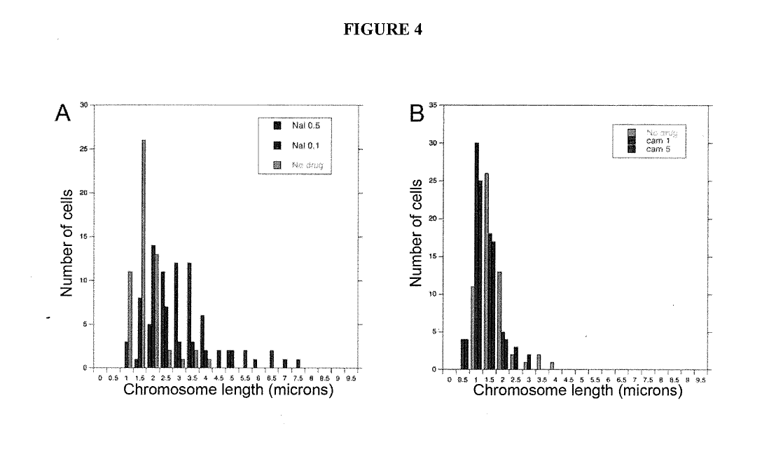

The shape of the chromosome is affected differently and in a dose dependent manner by different antibiotics (FIGS. 2 and 4). Cells treated with the gyrase inhibitor nalidixic acid have elongated chromosomes. In FIG. 4A, the chromosome length distribution for a population of untreated cells is compared to cells treated with two different concentrations of nalidixic acid for 2 hours. In untreated cells, the chromosomes ranged in length from 1 to 4 microns, whereas cells treated with a lethal dose of nalidixic acid ranged in length from 1 to 6 microns. Even when treated with levels of nalidixic acid five fold below the MIC, cells displayed very elongated chromosomes, ranging in length from 1 to 7.5 microns. This is not the case for cephalexin, in which the cells elongate, but chromosome shape is not affected (Pogliano et al. 1997).

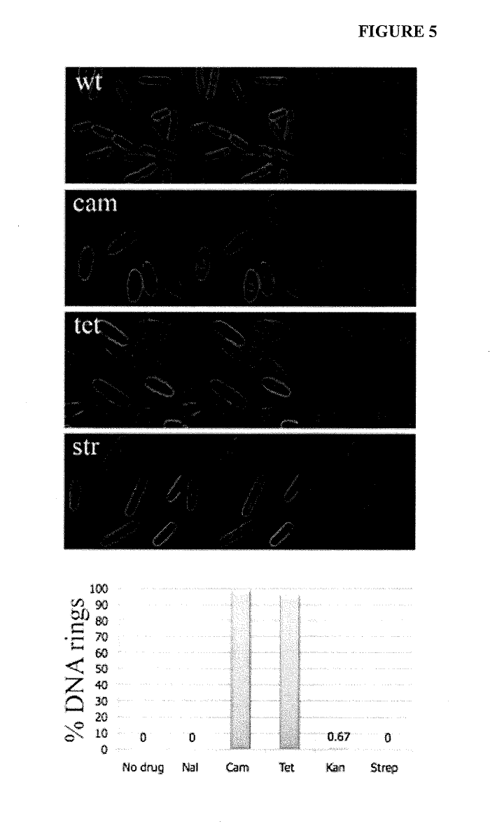

In contrast, the translational inhibitor chloramphenicol had a completely different effect on E. coli chromosome structure. After treating cells for two hours with either a lethal dose (5 μg/ml) or a sublethal dose (1 μg/ml), chromosomes were noticeably more condensed, ranging in length from 0.5 to 2 microns, in contrast to the 1 to 4 micron range of untreated chromosomes (FIG. 4B). The most striking effect was noted in the presence of a lethal dose of chloramphenicol and tetracycline. 98% of the chromosomes exhibited a striking doughnut or ring shape (FIG. 5). The doughnut shape was also present with high doses of chloramphenicol, such as 30 μg/ml, but not in nalidixic acid treated cells.

Other protein synthesis inhibitors were thus tested to determine if they would have a similar effect on the chromosome morphology. Fluorescence microscopy could distinguish between different protein synthesis inhibitors based on their mechanism of action. Both tetracycline and chloramphenicol caused chromosomes to form rings (FIG. 5). Although these two compounds are structurally different, they have a similar inhibitory effect on the ribosome (Wilson (2009)). Streptomcyin and Kanamycin, affect translation but act by a different mechanism. These compounds resulted in condensed chromosomes but not ring formation (FIG. 5). Thus, different protein synthesis inhibitors also result in different cytological patterns.

### E. Example 5

### Cytological Profiling can Detect the MOA of Antibiotic Agents at Concentrations Below the MIC (<⅕×MIC)

An advantage of cytological profiling (e.g., by microscopy) is the ability to observe striking cytological effects on cells that have virtually no effect on overall growth of the population. FIGS. 3 and 4A demonstrate that growth in the presence of a sublethal concentration of nalidixic acid results in elongated cells (FIG. 3) with extremely long chromosomes (FIG. 4A). In contrast, growth in the presence of a sublethal dose of chloramphenicol caused relatively subtle condensation of the chromosomes (FIG. 4B).

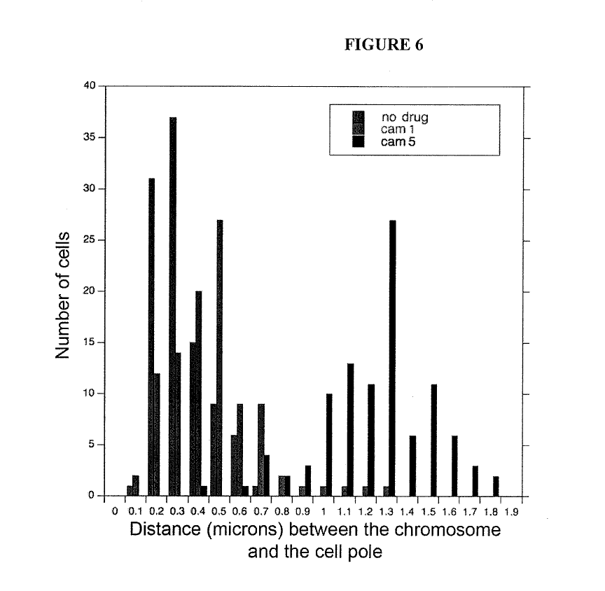

The distance between the edge of one chromosome and the pole of the cell is shown in FIG. 6. In untreated cells, the edges of the chromosomes were within 0.5 microns of the cell pole in nearly ⅔ of the cells, and this parameter ranged from 0.1 to 0.7 microns (FIG. 6; left bar). In cells treated with sublethal doses of chloramphenicol, this parameter increased significantly; the range of values extended from 0.1 micron up to 1.3 microns (FIG. 6; middle bar). As a control, this effect was measured after treatment with a lethal dose of chloramphenicol, which resulted in a dramatic increase (FIG. 6; right bar). Thus, additional parameters can be used to sensitively detect cytological effects of antibiotics even where there is little effect on overall growth.

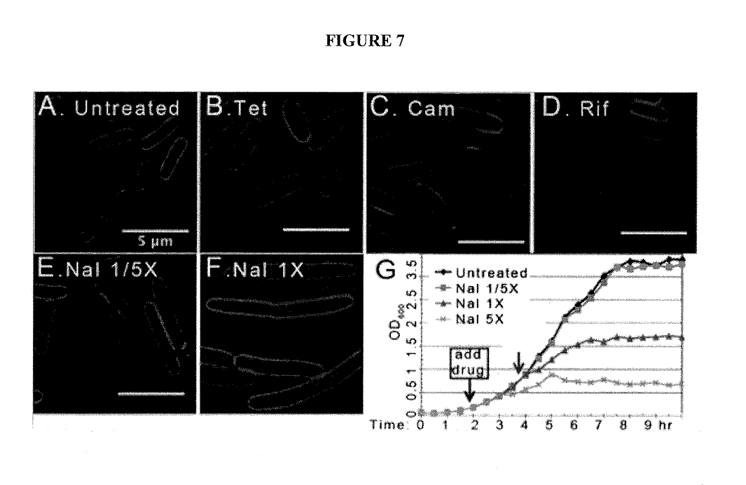

As a second example, nalidixic acid was added to cells at a lethal dose (1×MIC), resulting in reduced growth and dramatic morphological defects (FIG. 7). When nalidixic acid is added to a culture at a concentration five-fold below the MIC, there was no detectable effect on growth (based on optical density at 600 nm; FIG. 7G). However, low dose of nalidixic acid has a dramatic effect on cell morphology (FIG. 7E). The results show that cytological profiling is extremely sensitive for detecting the effects of multiple classes of antibiotics.

### F. Example 6

### Antibiotics that Target the Cell Envelope Result in Different Cytological Profiles

The above examples demonstrate distinction between the gyrase inhibitor nalidixic acid, the cell division inhibitor cephalexin, the protein synthesis inhibitors chloramphenicol and tetracycline, and the protein synthesis inhibitors streptomycin and kanamycin. The present example shows the unique cytological effects of other antibiotic classes, represented by vancomycin (binds the lipid II precursor for peptidoglycan biogenesis), penicillin G (inhibits cell wall biogenesis), nisin (assembles membrane channels), azide (reduces cellular ATP levels), and CCCP (a proton ionophore).

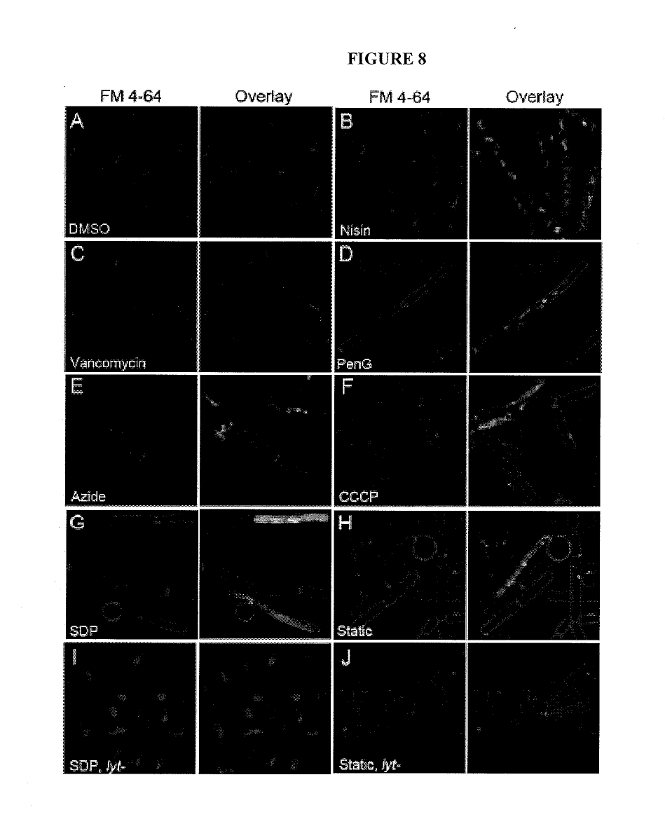

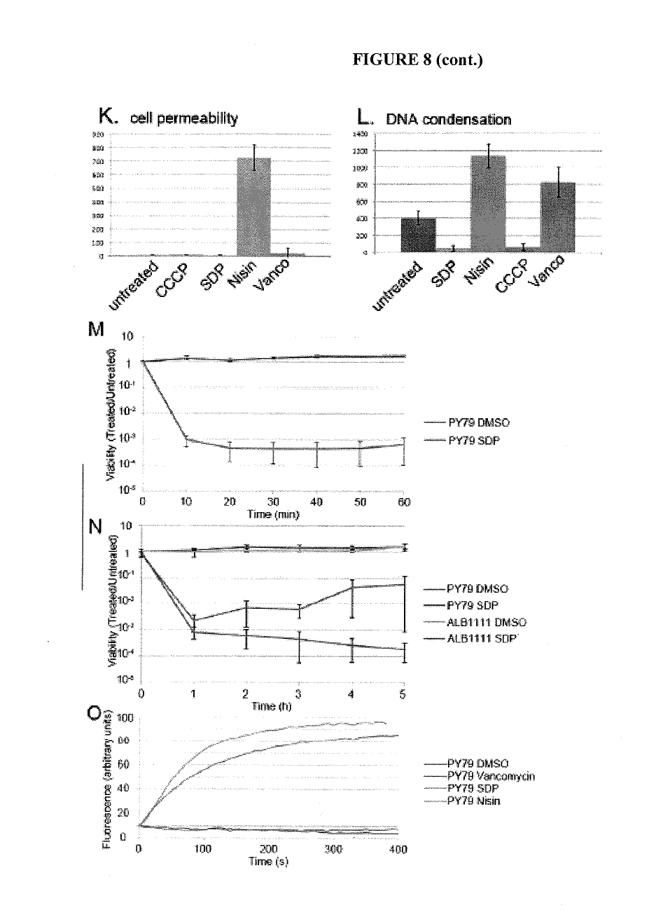

The effects of these drugs on the Gram positive bacterium Bacillus subtilis was determined using the fluorescent membrane stain FM 4-64 and two nucleotide stains that are membrane impermeable (DAPI and Sytox). The latter stains show low fluorescence intensity in intact cells (FIG. 8, untreated) and bright fluorescence on cells that either have permeabilized membranes or are completely lysed.

FIG. 8 shows that treatment with nisin, which causes large pores in the membrane, rapidly leads to increased DAPI and Sytox staining. Subtle membrane disruptions are also seen, such as uneven cell division and uneven staining of the cytoplasmic membrane. Treatment with vancomycin, which inhibits cell wall biogenesis by binding to the lipid II precursor, produces internal membrane vesicles and increased DAPI and Sytox permeability in a subset of the cells. Treatment with penicillin G, which inhibits cell wall biogenesis, produces cells with condensed chromosomes that lyse at various times. These lysed cells have large gaps in the cytoplasmic membrane and stain brightly with DAPI and Sytox. Treatment with two compounds that deplete cellular energy, sodium azide (which depletes cellular ATP) and the proton ionophore CCCP (which depletes the protein motive force (PMF)) produces cells with uneven membrane staining and decondensed chromosomes. Some of the cells lyse, showing large gaps in the membrane, greatly increased DAPI and Sytox staining, and extracellular membrane vesicles.

Cytological profiling can therefore distinguish between compounds that form large channels in the cytoplasmic membrane, those that disrupt cellular energy stores, and those that inhibit cell wall biogenesis, where it is further capable of discriminating between compounds that inhibit different steps in cell wall synthesis. Thus, cytological profiling is capable of reliably discriminating between compounds that affect different features of the cell envelope. These features can be measured using automated image analysis to allow cytological profiling to be employed in high throughput screens.

### G. Example 7

### Principal Component Analysis

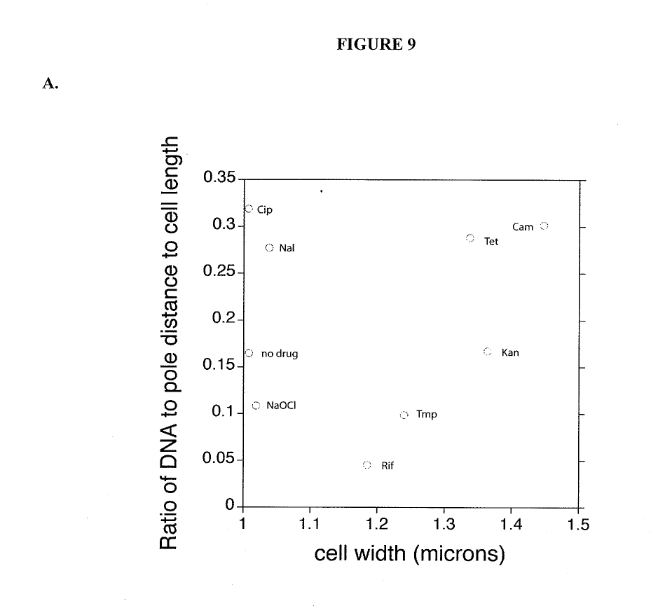

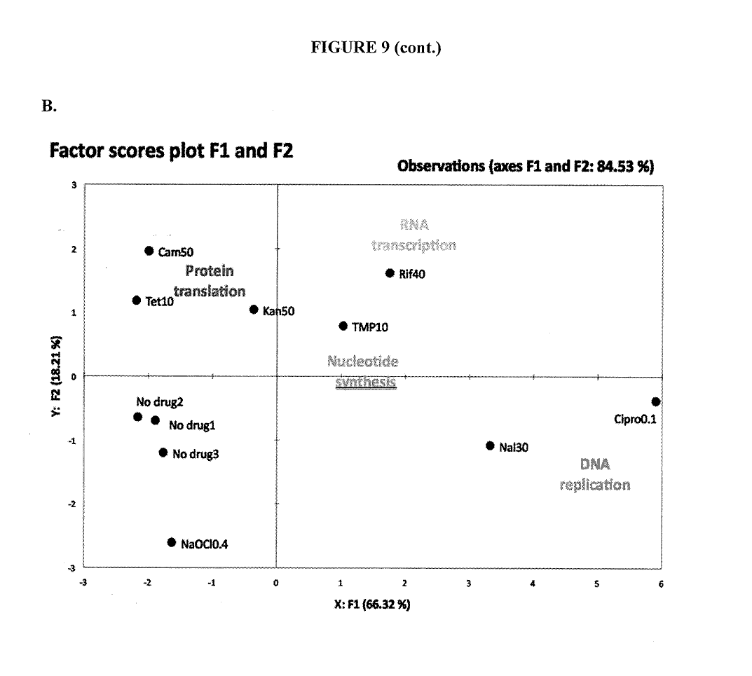

Cytological profiling relies upon making a series of measurements for each cell of the population. Two to more than a dozen different cytological measurements can be obtained. After making a series of measurements, the data can be analyzed in different ways. In the simplest type of analysis, two measurements are plotted against each other (see, e.g., FIG. 9A). FIG. 9A plots the average cell width versus the average of the ratio of the distance between the chromosome and the pole of the cell to average cell length. Using the two-dimensional plot, different antibiotics clearly separate into categories.

More than two factors can be used in the analysis to provide additional separation. Principle component analysis can thus also be used to analyze all of the cytological measurements made in a particular experiment. This powerful mathematical analysis allows for analysis of multiple cytological measurements, and identifies those most critical for discriminating between different antibiotic classes.

Principal component analysis (PCA) can be used to compare a large number of measurements and identify and quantify patterns in the data. The results are compressed from a multidimensional data set into a simplified 2D format (FIG. 9B). PCA can distinguish between compounds based on MOA, such as protein translation inhibitors, RNA transcription inhibitors, DNA replication inhibitors, proton motif force (PMF) inhibitors. These are further distinguished from untreated and bleach-treated (non-specific) control samples.

### H. Example 8

### Determination of MOA of Compounds where it is Unknown

Use of cytological profiling to determine the MOA of compounds where it is unknown, including newly identified compounds and one compound present in a crude extract is shown. The first compound, SDP, is the cannibalistic toxin of B. subtilis (Liu et al. (2010) PNAS 107:16286), which we found kills cells by rapidly depolarizing the membrane to trigger autolysis (FIG. 1). The second and third compounds, Spirohexenolide A and chlorothricin (Kang et al. (2009) J. Org. Chem. 74:9054; Pache & Champman (1972) Biochim Biophys Acta 255:348), are spirotetronate compounds active against gram positive bacteria and the E. coli imp mutant. The fourth compound, polymyxin B, is used as a treatment of last resort for drug resistant bacterial infections. Finally, cytological profiling was used to demonstrate that crude extracts containing bacillaene inhibit translation.

1. SDP

The cytological profile of SDP in FIG. 8 shows a dramatic lysis phenotype similar to that seen in azide and CCCP-treated cells and in non-aerated cultures. The phenotype indicates that SDP decreases cellular energy levels (either ATP or the proton motive force). The fluorescent stain DiSC₃(5) was used in a fluorometer assay, in which cells with a decreased PMF show increased fluorescence. This assay, as well as flow cytometry data, demonstrated that SDP (like Nisin and CCCP) rapidly depolarizes the membranes. SDP kills cells by depleting the PMF. Lysis is a secondary consequence of SDP treatment that occurs well after the loss of viability. Mutants lacking the major autolytic enzymes fail to lyse with SDP treatment, but are still rendered inviable. Thus, autolysis results from depletion of cellular energy stores.

Cytological profiling revealed key differences between the MOAs of Nisin and SDP. First, Nisin treated cells showed a rapid increase in permeability to Sytox and DAPI, likely due to the formation of large channels. SDP treated cells showed increased permeability only after lysis, indicating that SDP does not make large membrane channels. Second, Nisin treated cells did not show the dramatic lysis observed in SDP treated cells. In contrast, SDP treated cells are not rapidly permeabilized, maintaining a high internal osmotic pressure until autolytic enzymes digest the cell wall allowing for rapid extrusion of membrane vesicles.

The results show that cytological profiling can distinguish between cell lysing compounds that inhibit the PMF and those that make membrane channels.

2. Spirohexenolide A and Cholorothricin

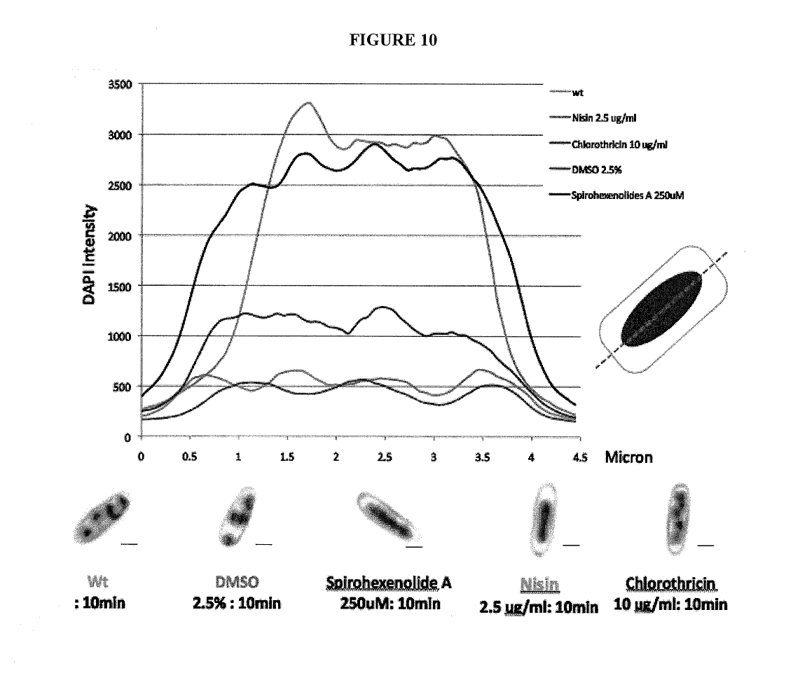

Cytological profiling was used to determine the MOA of spirohexenolide A. A culture of the E. coli imp strain was grown in LB media at 30° C. and spirohexenolide A was added at various concentrations. After either 10 minutes or two hours, samples were stained with FM 4-64 and DAPI for examination with a fluorescence microscope. The cytological profile of spirohexenolide A was identical to that for nisin, a compound that forms channels in the membrane. Chromosome stains such as DAPI and Sytox green are partially excluded from the cell by the cytoplasmic membrane in untreated cells, but are taken up in cells treated with permeablizers such as nisin. As shown in FIG. 10, cells treated with either nisin or spirohexenolideA stained 5× more intensely with DAPI than untreated (WT) cells. This suggests that spirohexenolide A permeabilizes the cytoplasmic membrane. Chlorothricin also increased DAPI staining, although to a somewhat lesser extent than Nisin or spirohexenolide A. The results show that spirohexenolide A and chlorothricin rapidly permeabilize cells in a manner similar to the pore-forming toxin nisin.

3. Polymyxin B

The polymyxins are antimicrobial agents for Gram negative bacteria that were originally described in the 1950's, but whose clinical use was discontinued due to dose-limiting toxicity (Zavascki et al. (2007) J. Antimicrob Chemother. 60:1206). The emergence of drug resistant Gram-negative infections has triggered the increased clinical use of polymyxin as the drug of last resort to treat infections caused by P. aeruginosa, A. baumannii, and K. pneumoniae. Toxicity issues remain troublesome, however, and often result in suspension of treatment.

The mechanism by which polymyxins kill cells is not fully understood. The widely accepted membrane disruption model is from in vitro studies, but the effect of polymyxins on living cells has not been determined.

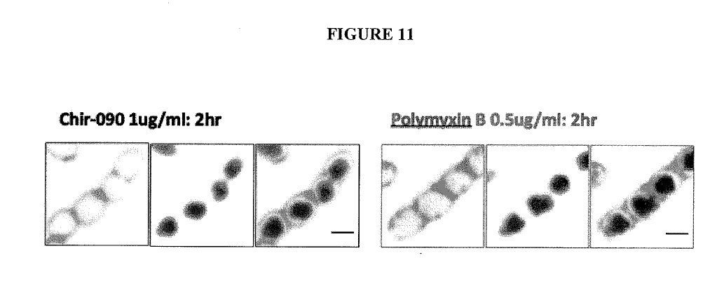

Cytological profiling with polymyxin B treated E. coli showed that polymyxin B shows a cytological profile similar to CHIR-090 (FIG. 11), a compound that inhibits LpxC (involved in synthesis of the lipid A moiety of LPS). The result indicates that polymyxin B blocks outer membrane biosynthesis (Barb et al. (2007) PNAS 104:18433). This new model indicates that polymyxin B acts by binding directly to lipid A and sequestering it, rather than simply using lipid A to bind the outer membrane prior to solubilizing it. This new model is consistent with the ability of polymyxin to sensitize Gram negative bacteria to vancomycin, a drug that normally kills only Gram-positive bacteria (Gordon et al. (2010) Antimicrob. Agents Chemother. 54:5316).

4. MOA Determination for Molecule in Crude Biological Extract

Natural products are an important source of antimicrobial compounds, but the complexity of crude extracts can make isolation difficult. Prior work with natural product extracts relied upon first purifying a potential new compound from the complex extract to homogeneity before attempting to determine the compound's identity and mechanism of action. (Molinari et al. (2009) Adv. Exp. Med. Biol. 655:13). The present results show that purification is not necessary, demonstrating the power of cytological profiling for antibiotic discovery.

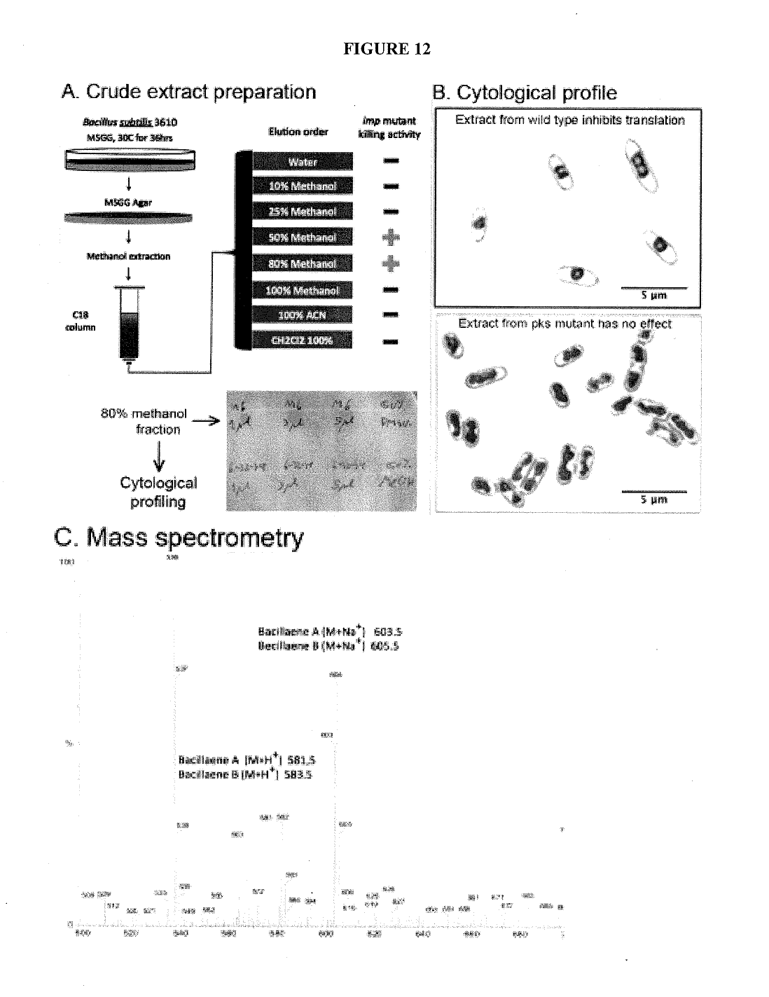

B. subtilis produces a variety of known antimicrobial compounds and its genome contains several uncharacterized pks genes that can produce uncharacterized compounds. An extract from B. subtilis strain 3610 was prepared (FIG. 12). The strain was first grown on a petri plate and then the agar and the bacteria were extracted with 95% methanol to remove extraneous natural products. The methanol extract was separated into eight fractions over a C18 column. Each of the fractions was screened for antibiotic activity by spotting several microliters (1 μl, 3 μl, or 5 μl) onto a lawn of an E. coli imp mutant (defective outer membrane). Two of the fractions (50% methanol and 80% methanol) contained antibiotic activity (indicated by clear zone).

Cytological profiling demonstrated that the extract contained an inhibitor of translation (FIG. 12). Based on these findings, the antibiotic activity was hypothesized to be the hybrid polyketide/non-ribosomal peptide bacillaene. Two experiments demonstrated that bacillaene was the translational inhibitor indicated by the cytological profile. First, an extract from a mutant 3610 strain (in the pks gene cluster required for bacillaene synthesis) was generated. Cytological profiling showed that extracts of the mutant strain neither inhibited translation (FIG. 12) or cell growth. Second, mass spectrometry showed that bacillaene is present in extracts that cause cell killing and inhibit translation (FIG. 12). Therefore, bacillaene is the antibiotic in the B. subtilis extracts.

5. Application of Bacterial Cytological Profiles to Eukaryotic Enzymes, Diseases, and Pathogens

Many essential biochemical pathways are conserved between bacterial and eukaryotic cells. Cytological profiling can be used to screen for drugs that are active against eukaryotic enzymes that have a functional homolog or analog in bacteria. In these screening methods, the bacterial enzyme is deleted and the eukaryotic enzyme is expressed in its place. For example, dihydrofolate reducate (DHFR) is essential for the synthesis of nucleotides in both bacteria and eukaryotes. DHFR inhibitors have been developed that are selective for either the bacterial enzyme (used as antibiotics), the Plasmodium malariae enzyme (used as antimalarials), or the mammalian enzyme (used as chemotherapeutics). Trimethoprim is an antibiotic that selectively inhibits bacterial DHFR. Methotrexate inhibits mammalian DHFR and is used to treat many types of cancer. Cytological profiling shows that trimethoprim treated cells have a unique cytological profile that is different from that of other antibiotics. Replacement of bacterial DHFR with Plasmodium DHFR or mammalian DHFR thus allows one to screen for antimalarial or anticancer drugs. A similar strategy may be applied to any human disease that relies on an enzyme with an essential counterpart in bacteria, and used to, e.g., screen for antifungal compounds, anti-malarial compounds, and anti-cancer drugs. Additional examples include other enzymes involved in DNA metabolism, amino acid metabolism, and mitochondrial function.

1-43. (canceled)

44. A method for determining a cytological profile of a microbial cell comprising:
(i) measuring one or more cytological characteristics of a microbial cell under a condition; and
(ii) correlating the one or more cytological characteristics with the condition.

45. A method for determining a cytological profile of a microbial cell comprising:
(i) measuring one or more cytological characteristics of a microbial cell under a first condition;
(ii) measuring said one or more cytological characteristics of a microbial cell under at least one additional condition; and
(iii) correlating the cytological characteristics with the conditions.

46. The method of claim 44 comprising measuring two or more cytological characteristics.

47. The method of claim 44 comprising measuring three or more cytological characteristics.

48. The method of claim 45 comprising measuring two or more cytological characteristics.

49. A method for identifying one or more agents that affect microbial cell cytological profiles comprising:
(i) exposing the microbial cell to one or more agents;
(ii) measuring one or more cytological characteristics of the microbial cell;
(iii) correlating the cytological characteristics with exposure to the one or more agents.

50. The method of claim 49 wherein the one or more agents inhibit microbial cell growth.

51. A method for identifying one or more cellular targets of one or more agents that affect microbial cell cytological profiles comprising:
(i) exposing the microbial cell to one or more agents;
(ii) measuring one or more cytological characteristics of the microbial cell;
(iii) comparing said measured cytological characteristic to one or more known cytological profiles; and
(iv) correlating the measured cytological characteristic of the agents with the known cytological profiles.

52. The method of claim 51 wherein the one or more known cytological profiles are generated by inhibiting a cellular pathway.

53. The method of claim 51 wherein the one or more known cytological profiles are generated by depletion of one or more cellular components.

54. A method for determining the susceptibility of a microbial cell to one or more potential therapeutic agents comprising:
(i) exposing the microbial cell to the one or more potential therapeutic agents;
(ii) measuring one or more cytological characteristics of the microbial cell; and
(iii) comparing the one or more cytological characteristics of the microbial cell with the cytological characteristics in microbial cells of the same strain not exposed to the one or more potential therapeutic agents.

55. A method for determining the susceptibility of a microbial cell to one or more potential therapeutic agents comprising:
(i) exposing the microbial cell to the one or more potential therapeutic agents;
(ii) measuring one or more cytological characteristics of the microbial cell; and
(iii) comparing the one or more cytological characteristics of the microbial cell with the cytological characteristics of susceptible strains exposed to the one or more potential therapeutic agents.

56. A method for determining the susceptibility of a microbial cell to one or more potential therapeutic agents comprising:
(i) exposing the microbial cell to the one or more potential therapeutic agents;
(ii) measuring one or more cytological characteristics of the microbial cell; and
(iii) comparing the one or more cytological characteristics of the microbial cell with the cytological characteristics of non-susceptible strains exposed to the one or more potential therapeutic agents.

57. A method for determining the strain of a microbial cell comprising:
(i) exposing the microbial cell to one or more agents;
(ii) measuring one or more cytological characteristics of the microbial cell; and
(iii) comparing the one or more cytological characteristics of the microbial cell with the cytological characteristics of known strains exposed to the one or more agents.

58. A method for evaluating the physiological state of a microbial cell comprising:
(i) measuring one or more cytological characteristics of the microbial cell; and
(ii) comparing the one or more cytological characteristics of the microbial cell with the cytological characteristics of the microbial cell under known conditions

59. A method for identifying an inhibitor of a eukaryotic protein comprising:
(i) expressing a heterologous eukaryotic protein in a microbial cell;
(ii) contacting the microbial cell with an agent;
(iii) measuring one or more cytological characteristics of the microbial cell; and
(iv) comparing the one or more measured cytological characteristics with the one or more measured cytological characteristics of the uncontacted bacteria.
